SEQUENCE LISTING (2 sequences)

SEQ ID NO: 1 - protein, 19 aa, artificial sequence
  targeting polypeptide ANG composed of L-type amino acids
TFFYGGSRGKRNNFKTEEY

SEQ ID NO: 2 - protein, 19 aa, artificial sequence
  trans-mirror sequence DANG of the targeting polypeptide composed of D-type amino acids
YEETKFNNRKGRSGGYFFT

(19) United States
(12) Patent Application Publication
(10) Pub. No.: US 2021/0338842 A1
(43) Pub. Date: Nov. 4, 2021

(54) DUAL-MODALITY NANOPROBE TARGETING GLIOBLASTOMA AND PREPARATION METHOD THEREOF

(71) Applicant: West China Hospital of Sichuan University, Chengdu City (CN)

(72) Inventors: Min WU, Chengdu City (CN); Qiyong GONG, Chengdu City (CN); Su LV, Chengdu City (CN); Fanxin ZENG, Chengdu City (CN); Ruoxi XIE, Chengdu City (CN)

(21) Appl. No.: 17/219,986

(22) Filed: Apr. 1, 2021

(30) Foreign Application Priority Data
May 3, 2020 (CN) 202010375037.8

(51) Int. Cl. A61K 49/18; A61K 49/00

(52) U.S. Cl. CPC A61K 49/1866; A61K 49/0002; B82Y 25/00; A61K 49/0056; A61K 49/0054

(57) ABSTRACT

A dual-modality nanoprobe targeting glioblastoma and preparation method thereof are described. The dual-modality nanoprobe of the present disclosure comprises DSPE-PEG(2000)-Amine, superparamagnetic iron oxide nanoparticles (SPIONs), Cy7-NHS molecule, targeting polypeptide and/or trans-mirror structure (i.e., enantiomer) thereof. The dual-modality nanoprobe can integrate the advantages of magnetic resonance and fluorescence imaging and thus provide clearer anatomical structure information of brain tumors; in the imaging process, the dual-modality nanoprobe can not only provide clearer image results, but also can specifically identify target sites. Also, the dual-modality nanoprobe of the present disclosure also has good in vivo stability.

### CROSS REFERENCE TO RELATED APPLICATION

This application claims the priority of Chinese Patent Application No. 202010375037.8, entitled “Dual-Modality Nanoprobe Targeting Glioblastoma And Preparation Method Thereof” filed with China National Intellectual Property Administration on May 3, 2020, which is incorporated herein by reference in its entirety.

### TECHNICAL FIELD

The disclosure relates to the technical field of biomedical materials, in particular to a dual-modality nanoprobe targeting glioblastoma and preparation method thereof.

### BACKGROUND ART

Due to the location specificity, heterogeneity and invasiveness of glioblastoma, it is necessary to obtain high spatial resolution and high sensitivity images before surgery to guide further surgery. However, the imaging diagnostic methods commonly used in clinic are difficult to achieve the above purpose.

On the one hand, penetration of blood-brain barrier (BBB) is considered as the main target of nano-drug or probe to transport to brain parenchyma. At present, in order to improve the plight of low accumulation of nano-delivery system in brain, researchers have proposed three delivery strategies, including bypassing BBB, destroying BBB and utilizing the endogenous transportation system of BBB. For example, BBB can be effectively bypassed by nasal injection, and drugs and nanoparticles can be directly transferred to the brain through olfactory nerve and trigeminal nerve without obvious peripheral exposure. However, due to the limited injection volume of this method, the content of drugs or nanoparticles entering the brain is low, thus leading low application efficiency. In addition, the BBB structure can be reversibly destroyed by physical methods such as focused ultrasound or magnetic field heating to achieve the purpose of drug delivery, but this method also allows other harmful substances to enter the brain. Different from the above two methods, which can only be realized with the support of external conditions, the third method is to use endogenous transporters or receptors in the brain to improve the efficient brain penetration of nanoparticles in a non-invasive manner, and modify related transporters, receptor-specific ligands or specific antibodies on the surface of nanoparticles, so that they can pass through BBB and target to tumor sites with high selectivity.

On the other hand, in the prior art, enhanced magnetic resonance imaging utilizing gadolinium chelate T₁ contrast agent is a commonly used clinical method for diagnosing glioblastoma, but Gd-based contrast agent still has many defects in the detection. Besides the leakage of blood vessels outside the tumor will have a great impact on enhanced imaging of T₁, it is difficult for enhanced imaging of T₁ itself to accurately understand postoperative reaction, especially in evaluating false positive progress after radiotherapy or false positive reaction during anti-angiogenesis therapy. In addition, Gd-based small molecule contrast agents, as the preferred contrast agents in MRI, still have some shortcomings, such as short imaging window time, poor enhancement effect, and potential nephrogenic fibrosis and nervous system deposition.

Therefore, it is necessary to develop new MRI contrast agents to eliminate the limitations of existing MRI contrast agents in clinical application. As T₂ contrast agent, superparamagnetic iron oxide nanoparticles (SPIONS) have the characteristics of high sensitivity, low toxicity and good biocompatibility, and are promising to be used in clinical research and application. However, due to the high replacement rate of cerebrospinal fluid, the close connection between brain endothelial cells and the low endocytosis efficiency of brain endothelial cells, more than 98% of small molecules and 100% of large molecules cannot enter the brain parenchyma, which seriously hinders the brain aggregation of nanoprobes and the brain delivery of nanodrugs.

Specific MR targeted contrast agents play an important role in molecular imaging research by combining with specific targets in vivo to show the status of molecular targets in vivo. Specific MR targeted contrast agent, also known as MR molecular probe, is a contrast agent developed on the basis of existing non-targeted contrast agents, which can reflect specific targeted molecules in the physiological or pathological process of human tissues, and can provide information on molecular level for early diagnosis and treatment of clinical diseases, as well as for the research on the pathogenesis of diseases. The specific MR targeted contrast agent are composed of carriers and imaging agents. Choosing specific carriers and good MR imaging agents for specific targets is the key factor to construct targeted molecular probes. Therefore, the combination of SPIONs and specific MR targeted contrast agent, i.e. the specific MR targeted contrast agent using SPIONs as imaging agent, is expected to overcome various obstacles of nanoprobe brain delivery, and then obtain good MRI images of intracranial lesions, thus providing guidance for the diagnosis and treatment of glioblastoma.

At the same time, the single imaging mode has obvious limitations, such as MRI is limited by low sensitivity, and optical imaging is affected by low spatial resolution and lack of tissue permeability. However, the combined dual-modality imaging can provide collaborative and complementary imaging information and achieve complementary advantages. Therefore, using magnetic resonance/optical dual-modality nanoprobes to combine magnetic resonance contrast agents with near infrared fluorescent molecules (Cy5.5, Cy7, etc.) is an important development direction of specific MR targeted contrast agents in the future.

### SUMMARY OF THE INVENTION

A purpose of the present disclosure is to provide a dual-modality nanoprobe targeting glioblastoma and preparation method thereof, which solves the technical problem that a single imaging mode of the nanoprobe in the prior art is difficult to obtain clear image results. Many technical effects produced by the preferred technical scheme of the present disclosure are described in detail below.

In order to achieve the above purpose, the present disclosure provides the following technical solution:

A dual-modality nanoprobe targeting glioblastoma in the present disclosure, comprising: DSPE-PEG(2000)-Amine, superparamagnetic iron oxide nanoparticles (SPIONs), Cy7-NHS molecule, targeting polypeptide and/or trans-mirror structure (i.e., enantiomer) thereof. In some embodiments, the DSPE-PEG(2000)-Amine has a number-average molecular weight (Mn) of 2742.

In a preferred embodiment, the targeting polypeptide is a targeting polypeptide ANG composed of L-type amino acids; the trans-mirror structure (i.e., enantiomer) of the targeting polypeptide is the trans-mirror (i.e., enantiomer) sequence ^{D}ANG of the targeting polypeptide composed of D-type amino acids.

In a preferred embodiment, the targeting polypeptide ANG composed of L-type amino acids comprises the sequence of Ac-TFFYGGSRGKRNNFKTEEY-OH (SEQ ID NO: 1); the trans-mirror (i.e., enantiomer) sequence ^{D}ANG of the targeting polypeptide composed of D-type amino acids comprises the sequence of Ac-YEETKFNNRKGRSGGYFFT-OH (SEQ ID NO: 2).

Without being limited to this, the targeting polypeptide and/or trans-mirror structure (i.e., enantiomer) thereof in the present disclosure can also be other sequences.

In a preferred embodiment, the dual-modality nanoprobe is Peptides/Cy7-PEG-DSPE-SPIONs, and the Peptides/Cy7-PEG-DSPE-SPIONs comprise ANG/Cy7-PEG-DSPE-SPIONs and/or ^{D}ANG/Cy7-PEG-DSPE-SPIONs.

In a preferred embodiment, the dual-modality nanoprobe is ^{D}ANG/C_{y}7-PEG-DSPE-SPIONs.

A preparation method of the dual-modality nanoprobe targeting glioblastoma according to any one of the technical solutions in the present disclosure, comprising: using superparamagnetic iron oxide nanoparticles (SPIONs) as a core, modifying the surface of SPIONs with near infrared fluorescent molecule Cy7, and taking the targeting polypeptide and/or trans-mirror structure (i.e., enantiomer) thereof as ligands to construct magnetic resonance/fluorescence dual-modality nanoprobe targeting glioblastoma.

In a preferred embodiment, the preparation method of the dual-modality nanoprobe targeting glioblastoma, comprising:

S1, preparing SPIONs, coating DSPE-PEG(2000)-Amine molecules on the surface of the SPIONs through hydrophilic-hydrophobic interaction, and carrying out ultrasonic dispersion pretreatment on pegylated magnetic nanoparticles having amino groups to uniformly disperse the nanoparticles in an aqueous solution, replacing the aqueous solution of magnetic nanoparticles by 0.02 M, pH8 borate buffer solution with a 10 KDa ultrafiltration tube;

S2, activating carboxyl groups of the targeting polypeptide ANG composed of L-type amino acids and/or the trans-mirror (i.e., enantiomer) sequence ^{D}ANG of the targeting polypeptide composed of D-type amino acid;

S3, placing the activated targeting polypeptide ANG and/or the trans-mirror (i.e., enantiomer) sequence ^{D}ANG of the targeting polypeptide composed of D-type amino acid and NH₂-PEG-DSPE-SPIONs on a shaking table to react for 2 hours, and separating the unreacted targeting polypeptide by a 30 KDa ultrafiltration tube to obtain a pegylated magnetic nanoparticle Peptides/PEG-DSPE-SPIONs solution;

S4, adding Cy7-NHS molecules into the pegylated magnetic nanoparticle solution, and shaking overnight at 4° C. in the dark for incubation; after the incubation, removing the unreacted Cy7-NHS dye molecule by ultrafiltration tube to obtain a Peptides/Cy7-PEG-DSPE-SPIONs probe.

In a preferred embodiment, the SPIONs are synthesized by hydrothermal method. Preferably, the hydrothermal synthesis of SPIONs comprises:

S11, mixing iron acetylacetonate, 1, 2-hexanediol, oleic acid, oleamide and benzyl ether and putting into a round bottom flask, passing argon to remove air in the reaction system and stirring to obtain a mixture;

S12, heating the mixture to 200° C., keeping constant temperature for 2 h, then heating to 300° C., refluxing for 1 h to obtain a black mixture;

S13, cooling the black mixture to room temperature, precipitating the product with ethanol, centrifuging to remove solvent, and dispersing the product to hexane to obtain a SPIONs solution;

S14, centrifuging the SPIONs solution to remove aggregates.

In a preferred embodiment, activating the carboxyl groups of the targeting polypeptide ANG composed of L-type amino acids and/or the trans-mirror (i.e., enantiomer) sequence ^{D}ANG of the targeting polypeptide composed of D-type amino acid comprises:

S21, dissolving the targeting polypeptide ANG composed of L-type amino acids and/or the trans-mirror (i.e., enantiomer) sequence ^{D}ANG of the targeting polypeptide composed of D-type amino acid with 0.02 M MES buffer solution with a pH of 5.5;

S22, adding EDC molecules and NHS molecules, reacting for 25 min at 25° C. and 180 rpm in a constant temperature shaker to activate carboxyl groups of the targeting polypeptide sequence;

S23, removing unreacted EDC and NHS with 2 KDa ultrafiltration tube.

The dual-modality nanoprobe targeting glioblastoma and the preparation method thereof provided by the disclosure have at least the following beneficial effects:

The dual-modality nanoprobe of the present disclosure comprises DSPE-PEG(2000)-Amine, superparamagnetic iron oxide nanoparticles (SPIONs), Cy7-NHS molecule, targeting polypeptide and/or trans-mirror structure (i.e., enantiomer) thereof The dual-modality nanoprobe can combine the advantages of magnetic resonance and fluorescence imaging and thus providing clearer anatomical structure information of brain tumors; in the imaging process, the dual-modality nanoprobe can not only provide clearer image results, but also can specifically identify target sites. That is, by constructing the magnetic resonance/fluorescence dual-modality nanoprobe capable of targeting glioblastoma, the disclosure solves the technical problem that a single imaging mode of the nanoprobe is difficult to obtain clear image results in the prior art;

On the other hand, the magnetic resonance/fluorescence dual-modality nanoprobe constructed by the disclosure can realize the MRI enhanced imaging function, and at the same time, endows the probe with near infrared fluorescence imaging function, especially the dual-modality nanoprobe ^{D}ANG/Cy7-PEG-DSPE-SPIONs, which are constructed by using the trans-mirror (i.e., enantiomer) sequence of targeting polypeptide composed of D-type amino acids, has better in vivo stability.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In order to explain the embodiments of the present disclosure or the technical scheme in the prior art more clearly, the drawings required in the embodiments or the description of the prior art will be briefly introduced below. Obviously, the drawings in the following description are only some embodiments of the present invention, for those of ordinary skill in the art, other embodiments can be obtained based on these drawings without creative work.

FIG. 1A and FIG. 1B are the model diagram and physical diagram of BBB model constructed by Transwell in Example 1, respectively; FIG. 1C is the NaFl standard curve diagram after BBB was constructed in vitro in Example 1; FIG. 1D is the immunofluorescence staining diagram of endothelial interstitial protein ZO-1 protein in Example 1;

FIG. 2A and FIG. 2B are flow cytometry diagrams showing the uptake of ANG/Cy7-PEG-DSPE-SPIONs and ^{D}ANG/Cy7-PEG-DSPE-SPIONs by bEnd.3 cells in Example 3, respectively; FIGS. 2C and 2D are flow cytometry diagrams showing the uptake of ANG/Cy7-PEG-DSPE-SPIONs and ^{D}ANG/Cy7-PEG-DSPE-SPIONs by U87-MG cells in Example 3, respectively; FIG. 2E shows the quantitative result of the uptake of ANG/Cy7-PEG-DSPE-SPIONs by bEnd.3 cells and U87-MG cells in Example 3; FIG. 2F shows the quantitative result of the uptake of ^{D}ANG/Cy7-PEG-DSPE-SPIONs by bEnd.3 cells and U87-MG cells in Example 3;

FIG. 3A and FIG. 3B are flow cytometry diagrams showing the uptake of ^{D}ANG/C_{y}7-PEG-SPIONs and ANG/C_{y}7-PEG-SPIONs probes (treated (incubated)/untreated with serum) by U87-MG cells in Example 5;

FIG. 4 shows the quantitative result indicating the uptake of ^{D}ANG/C_{y}7-PEG-SPIONs and ANG/C_{y}7-PEG-SPIONs probes (after treated (incubated) with serum) by U87-MG cells in Example 5;

FIG. 5 is a near infrared fluorescence image that depicts results of Luc-U87-MG in situ glioblastoma model in nude mice, wherein mouse A is an unsuccessful model, and mice, B, C, and D are successful model diagrams.

FIG. 6 is a magnetic resonance imaging diagram of Cy7-PEG-DSPE-SPIONs probe (control) in Example 8 in tumor-bearing nude mice at different time points, wherein the upper row is T₂ weighted imaging diagram and the lower row is magnetic sensitivity weighted imaging (SWI, T₂*) diagram;

FIG. 7 is a magnetic resonance imaging diagram of ANG/Cy7-PEG-DSPE-SPIONs probes in Example 8 in tumor-bearing nude mice at different time points, wherein the upper row is T₂ weighted imaging diagram and the lower row is magnetic sensitivity weighted imaging (SWI, T₂*) diagram;

FIG. 8 is a magnetic resonance imaging diagram of ^{D}ANG/C_{y}7-PEG-DSPE-SPIONs probes in Example 8 in tumor-bearing nude mice at different time points, wherein the upper row is T₂ weighted imaging diagram and the lower row is magnetic sensitivity weighted imaging (SWI, T₂*) diagram;

FIG. 9 is an in vitro near infrared fluorescence imaging of Cy7-PEG-DSPE-SPIONs probe (control), two kinds of Peptides/Cy7-PEG-DSPE-SPIONs probes (experimental groups) injected into tumor-bearing nude mice for 24 h, wherein the upper row is a bioluminescent imaging showing tumor size; the lower row is a near infrared fluorescence imaging showing the probe position;

FIG. 10 is a comparison graph of the average fluorescence intensity of the three probes in Example 9 at the glioblastoma;

FIG. 11 is a distribution diagram of the three probes in Example 10 in the brain and tumor position, wherein the lower row is an enlarged picture of the upper row, and the scale of upper row is 50 μm, the scale of lower row is 25 μm;

FIG. 12A and FIG. 12B show the detection results of toxic and side effects of Peptides/Cy7-PEG-DSPE-SPIONs probes in Example 12 on U87-MG and HUVEC respectively;

FIG. 13 is a graph of HE staining of main organs 1 day after the tail vein injection of three probes in Example 13, and the scale in FIG. 13 is 50 μm;

FIG. 14 is a graph of HE staining of main organs 3 days after the tail vein injection of three probes in Example 13, and the scale in FIG. 13 is 50 μm;

FIG. 15 is a graph of HE staining of main organs 7 days after the tail vein injection of three probes in Example 13, and the scale in FIG. 13 is 50 μm.

### DETAILED DESCRIPTION OF THE EMBODIMENTS

In order to make the purpose, technical scheme and advantages of the present disclosure clearer, the technical scheme of the present disclosure will be described in detail below. Obviously, the described embodiments are only part of the embodiments of the present invention, not all of them. Based on the embodiments of the present invention, all other embodiments obtained by those of ordinary skill in the art without creative labor shall fall within the protection scope of the present disclosure.

The preparation method of the dual-modality nanoprobe targeting glioblastoma comprises:

S1, synthesizing SPIONs by hydrothermal method and pretreating the SPIONs as follows:

2 mmol of iron acetylacetonate, 10 mmol of 1, 2-hexanediol, 6 mmol of oleic acid, 6 mmol of oleamide and 20 mL of benzyl ether are mixed and put into a round bottom flask, and argon is passed to remove air in the reaction system and the mixture is stirred. The mixture is heated to 200° C. and the temperature is maintained for 2 h, then heated to 300° C. and refluxed for 1 h to obtain a black mixture. The black mixture is cooled to room temperature, precipitated with ethanol, and the precipitation is centrifuged (10,000 g, 10 min) to remove the solvent, and a SPIONs solution is obtained by dispersing the product to hexane. The obtained SPIONs solution is centrifuged at 3,000 g to remove aggregates.

DSPE-PEG(2000)-Amine molecule is coated on the surface of SPIONs by hydrophilic-hydrophobic interaction, and the pegylated magnetic nanoparticles with amino groups are pretreated by ultrasonic dispersion, so that the nanoparticles are uniformly dispersed in aqueous solution. The aqueous solution of magnetic nanoparticles is replaced by 0.02 M, pH 8 borate buffer solution with a 10 KDa ultrafiltration tube.

S2, activating the carboxyl of the targeting polypeptide, specifically as follows:

the carboxyl of targeting polypeptide ANG composed of L-type amino acid and/or trans-mirror (i.e., enantiomer) sequence ^{D}ANG composed of D-type amino acid are activated, comprising:

2 mg of targeting polypeptide ANG composed of L-type amino acids and/or trans-mirror (i.e., enantiomer) sequence ^{D}ANG composed of D-type amino acids are taken and dissolved in 0.02 M MES buffer solution pH 5.5;

1 mg EDC molecule and 0.5 mg NHS molecule is added, and reacted for 25 min at 25° C. and 180 rpm on a constant temperature shaker to activate the carboxyl group of the targeting polypeptide sequence, NHS molecules function for connecting;

unreacted EDC and NHS are removed by 2 KDa ultrafiltration tube (3000 rpm, 5 min, centrifugation twice).

S3, placing the activated targeting polypeptide ANG and/or the trans-mirror (i.e., enantiomer) sequence ^{D}ANG of the targeting polypeptide composed of D-type amino acid and NH₂-PEG-DSPE-SPIONs on a shaking table to react for 2 hours, and separating the unreacted targeting polypeptide by a 30 KDa ultrafiltration tube to obtain a pegylated magnetic nanoparticle Peptides/PEG-DSPE-SPIONs solution.

S4: Cy7-NHS molecule (50 μL, 1 mg/mL) is added into the pegylated magnetic nanoparticle solution, and oscillated overnight at 4° C. in the dark for incubation; after the incubation, the unreacted dye molecule Cy7-NHS is removed by ultrafiltration tube to obtain Peptides/Cy7-PEG-DSPE-SPIONs probe.

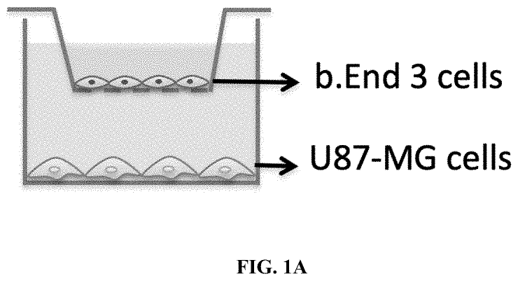

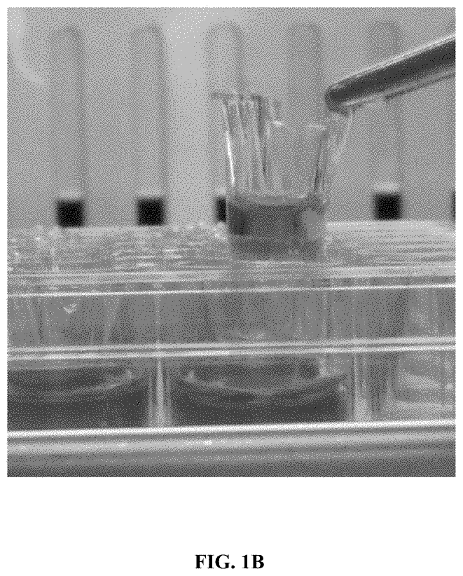

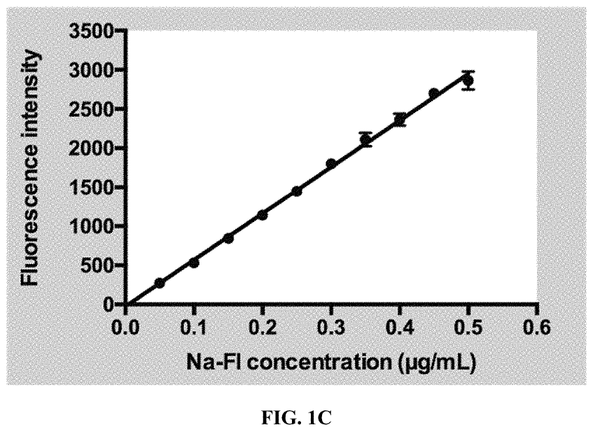

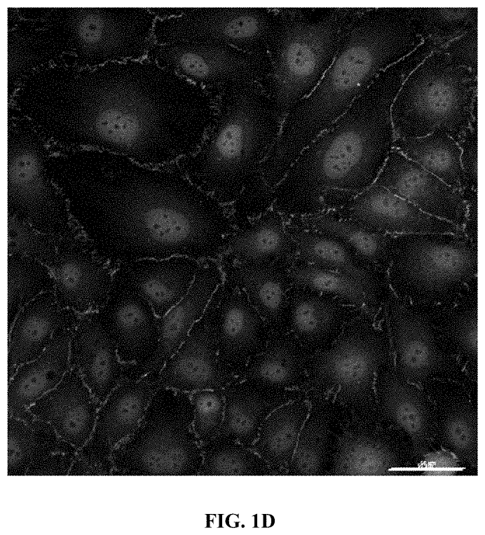

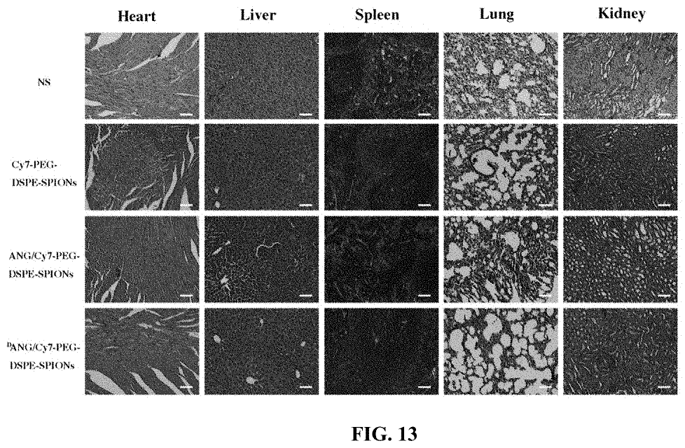

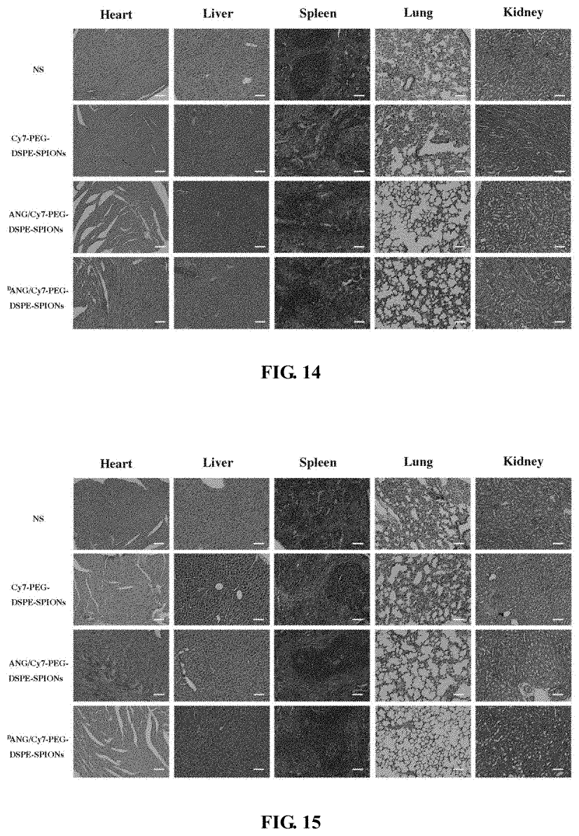

Taking targeting polypeptide ANG composed of L-type amino acids and trans-mirror (i.e., enantiomer) sequence ^{D}ANG of the targeting polypeptide composed of D-type amino acids as examples, in conjunction with FIGS. 1-15 and Examples 1-13, the dual-modality nanoprobe for targeting glioblastoma prepared by the method of the present disclosure is described in detail through experiments of BBB penetration characteristics of contrast agents in vitro, cell imaging in vitro and in vivo. The data in the experiment is statistically analyzed by Prism 6 software, and the experimental results are expressed as mean±standard deviation. The differences between the two groups are compared by one-way ANOVA, and the difference is judged to be significant when p<0.05.

### Experimental Materials

TABLE 1
main material reagent

| Name | Manufacturer |
|---|---|
| DSPE-PEG(2000)-Amine, Mn = 2742 | Nanjing Nanoeast Biotech |
| | Co., Ltd |
| Polypeptide (ANG, ^{D}ANG) | Zhejiang Ontores Biotech |
| | Co., Ltd |
| Serum | GEMINI, USA |
| Penicillin/streptomycin | HyClone, USA |
| High sugar dmem medium | HyClone, USA |
| 0.25% Trypsin | HyClone, USA |
| PBS | HyClone, USA |
| Collagen | StemCell, CA |
| ZO-1 antibody | Invitrogen, USA |
| 4% Paraformaldehyde | Beyotime biotech, CN |
| BSA | Solarbio, CN |
| FITC Second antibody | Beyotime biotech, CN |
| DAPI | Beyotime biotech, CN |
| Fluorescein sodium(NaFl) | Sigma, USA |
| Puromycin | Solarbio, CN |
| Potassium fluorescein salt | Beyotime biotech, CN |
| Isoflurane | RWD biotech, CN |
| Sucrose | Kelong biotech, CN |
| OCT embedding agent | SAKURA, USA |
| Prussian blue solution A (2% HCl), | Yuanye biotech, CN |
| solution B (2% potassium | |
| ferrocyanide solution) | |
| Neutral resin | Kelong biotech, CN |
| H₂O₂ | Kelong biotech, CN |
| HNO₃ | Kelong biotech, CN |
| Transwell | Millipore, USA |
| 96-well black ELISA plate | Corning, USA |
| 24-well plates | Thermo, USA |
| 12-well plates | Thermo, USA |
| 96-well plates | Thermo, USA |
| Circular glass | CITOTEST ®, CN |
| 5 μL Microinjector | RWD biotech, CN |
| Medical suture | Johnson & Johnson, USA |
| Cationic anti-deloading slides | CITOTEST ®, CN |
| 15 mL centrifuge tube | BD, USA |
| 10 cm cell culture dish | Thermo, USA |
| CCK-8 kit | Dojindo, JP |

TABLE 2
main equipment

| Instruments | Model | |
|---|---|---|
| and equipments | specification | Manufacturer |
| 3.0 T Medical nuclear magnetic | MAGNETOM Trio | Simens, DE |
| resonance imager | | |
| Small animal living imager | IVIS Spectrum | Caliper Life |
| | | Sciences, USA |
| Multifunctional ELISA reader | Synergy Mx | BioTek, USA |
| Confocal laser scanning | A1RMP + | Nikon, JP |
| microscope | | |
| Flow cytometry | FACSAria | BD, USA |
| Optical microscope | AX10 imager A2 | Zeiss, DE |
| Constant cold microtome | CM3050S | Leica, DE |
| Inductively coupled plasma | VG PQExCell | TJA, USA |
| mass spectrometry (ICP-MS) | | |
| ELISA reader | EON | BioTek, USA |

(3) Cells

bEnd.3: mouse cerebral vascular endothelial cell line, purchased from Shanghai cell bank of Chinese Academy of Sciences, and stored in laboratory with liquid nitrogen.

U87-MG: astrocytoma of human brain cell line, purchased from Shanghai Cell Bank of Chinese Academy of Sciences, and stored in laboratory with liquid nitrogen.

(4) Animals

BAL B/c mice, male, 20±2 g, provided by Chengdu Dashuo Experimental Animal Center.

BAL B/c nude mice, male, 20±2 g, provided by Chengdu Dashuo Experimental Animal Center.

### EXAMPLE 1

This Example illustrates the construction and evaluation of BBB model in vitro.

Experimental method: an in vitro blood-brain barrier model was established to evaluate the permeability of dual-modality nanoprobes. U87-MG cells and bEnd.3 cells were cultured in high glucose DMEM containing 10% fetal bovine serum and 1% penicillin/streptomycin at 37° C. and 5% CO₂. A thin layer of collagen solution was evenly coated on the inner side of Transwell poly microporous lipid membrane, the membrane was placed on an ultra-clean table for about 30 min, and it was used after naturally dried. bEnd.3 cells were inoculated into the upper chamber of a 24-well Transwell at a density of 5×10⁴ cells/well. Two days later, U87-MG cells were inoculated into the lower chamber of the upper chamber at a density of 1×10⁵ cells/well, and the two kind of cells were continued to co-culture for one week.

Firstly, the BBB model was tested and evaluated by leak test. The upper and lower chambers of Transwell were added with 200 μL and 900 μL culture medium respectively, and the liquid level difference was recorded. After 4 hours of continuous culture, the liquid level difference between the upper and lower chambers was observed.

The tight junction protein ZO-1 was expressed on the endothelial cell membrane and in a continuous linear distribution. The tight junction between the epithelial cells in the upper chamber can be judged by the immunohistochemical identification of ZO-1. The media in the upper and lower chamber of Transwell was sucked off, washed twice with PBS, and the polyester film was cut off and put into a 24-well plate for immunofluorescence staining. 4% paraformaldehyde was added to fix cells for 30 min, the Transwell was washed with PBS for 3 times, added with 5% BSA solution, and sealed for 1 h at room temperature. ZO-1 rabbit primary antibody (5 μg/mL) was added and incubated at 4° C. overnight. After removing the primary antibody, the Transwell was washed with PBS 3 times, FITC labeled goat anti-rabbit secondary antibody was added, and the membrane was incubated for 1 h at 37° C. After washing with PBS 3 times, DAPI was added to stain the nucleus, and a sample preparation after washing was obtained for confocal microscopy.

The permeability of BBB in vitro was evaluated by fluorescein sodium, and a series of standard solutions of fluorescein sodium (NaFl) were prepared with PBS, with the concentrations of 0.05, 0.10, 0.15, 0.20, 0.25, 0.30, 0.35, 0.40, 0.45 and 0.50 μg/mL, respectively. The absorbance value was measured with a multifunctional ELISA reader (the excitation wavelength was set at 485 nm and the detection wavelength was 535 nm). Then the standard curve of NaFl concentration and absorbance value was made. The BBB model constructed above was taken, the culture medium was sucked from the upper and lower chamber of Transwell, and the Transwell washed with PBS twice, 200 μL NaFl (10 μg/mL) solution was added in the upper chamber, 900 μL PBS was added in the lower chamber, and the chambers were cultured at 37° C. and 5% CO₂ respectively for 30 min, 60 min and 90 min, then 100 μL of the chamber solution was taken to the 96-well plate, and the lower chamber was replenished to 900 μL every time, the amount of NaFl passing through the upper chamber was determined by multifunctional ELISA. The permeability coefficient Papp was calculated by the formula: Papp (cm/s)=dQ/dt×1/(A×C₀), wherein dQ/dt is the transport rate of NaFl from the upper chamber to the lower chamber of Transwell, A is the diffusion area, which is 0.33 cm² in this experiment, and C₀ is the initial concentration of the drug in the upper chamber.

Experimental results: the construction of BBB model in vitro is shown in FIG. 1A and FIG. 1B. b. End3 cerebral vascular endothelial cells was added in the upper chamber of Transwell, U87-MG glioblastoma cells was added in the lower chamber, and BBB simulated barrier was formed after co-culture.

Leak test: after the model is constructed, the fluid is changed, and the model is observed after 4 hours. The liquid level difference between the upper chamber and lower chamber of Transwell small chamber keeps relatively stable. It can be preliminarily considered that the epithelial cells in the upper chamber were completely and tightly connected, and the BBB model is basically formed.

Immunohistochemical identification of tight junction protein ZO-1 in mouse cerebral vascular endothelial cells: there is tight junction among b. End3 cells, and tight junction protein ZO-1 is expressed on the surface of cell membrane, showing continuous linear distribution. The results of immunofluorescence detection were shown as FIG. 1D, and the expression of ZO-1 protein among endothelial cells can be observed.

Evaluation of BBB restricted permeability: the standard curve of NaFl concentration determination is y=6165×+15.76, r²=0.9985, x is the concentration and y is the absorbance value (as shown in FIG. 1C). The NaFl concentration in the upper chamber was measured at different time points of 30 min, 60 min and 90 min. The NaFl transmittance of the small blank chamber is calculated: Papp=67.80×10⁻⁶ cm/s, and BBB model group: Papp=(11.24±0.73)×10⁻⁶ cm/s. This result shows that BBB model has a strong ability to limit permeability.

### EXAMPLE 2

In this Example, the performance of dual-modality nanoprobe penetrating in vitro BBB model was evaluated.

Experimental method: after successfully constructing BBB model in vitro by the method of Example 1, the upper and lower chambers of Transwell were gently cleaned twice with PBS, and then PBS, Cy7-PEG-DSPE-SPIONs and two kinds of peptide/Cy7-PEG-DSPE-SPIONs solutions (the two kinds of peptide/Cy7-PEG-DSPE-SPIONs solutions were ANG/Cy7-PEG-DSPE-SPIONs and ^{D}ANG/Cy7-PEG-DSPE-SPIONs; similarly, the two peptide/Cy7-PEG-DSPE-SPIONs solutions mentioned in the following examples will also be ANG/Cy7-PEG-DSPE-SPIONs and ^{D}ANG/Cy7-PEG-DSPE-SPIONs, without repeated in details)were added into the upper chamber, and the concentration of each group is 10.0 μg/mL, with a total of 200 μL; 900 μL PBS was added into the lower chamber, and incubated for 60 min, 100 μL of buffer solution in the lower chamber was added into PET black 96-well plate, the fluorescence intensity of the lower chamber solution was measured three times with multi-functional ELISA, and the amount of probes loaded was calculated, with the excitation wavelength of 720 nm and detection wavelength of 820 nm.

Experimental results: Cy7-PEG-DSPE-SPIONs is taken as a control to evaluate the penetration ability of the two kinds of Peptides/Cy7-PEG-DSPE-SPIONs probes in vitro BBB models by measuring the probe solution penetrating through the upper chamber. After adding equal amounts of ANG/Cy7-PEG-DSPE-SPIONs and ^{D}ANG/C_{y}7-PEG-DSPE-SPIONs to the upper chamber of Transwell for 60 min, the fluorescence intensity of the probe passing through the upper chamber is 624.6±41.6 and 674±62.22, respectively, there is a significant difference (p<0.05) between the experimental group and the control group (CY7-PEG-DSPE-SPIONS, 158±47.53), indicating that the BBB penetration ability of the probe has significantly improved after modification of the targeting polypeptide.

### EXAMPLE 3

In this Example, the uptake of different probes by bEnd.3 cells and U87-MG cells is quantitatively analyzed.

Experimental method: cell plating: U87-MG cells and bEnd.3 cells were cultured with high glucose DMEM containing 10% fetal bovine serum and 1% penicillin/streptomycin at 37° C. and 5% CO₂. When the cells grew well, the two kinds of cells were spread in 12-well plates with a density of 1×10⁵ cells per well.

Flow cytometry: after the cells adhered to the wall, two probe solutions (Peptides/Cy7-PEG-DSPE-SPIONs) with a concentration of 50 μg/mL (Fe₃O₄ concentration) were added, the cells cultured at 37° C. and 5% CO₂ respectively for 1 h, 2 h and 4 h, the cells were collected for flow cytometry at the set time points, and the detection channel was set as APC-A750, the data results were processed and analyzed by flow cytometry software Flowjo.

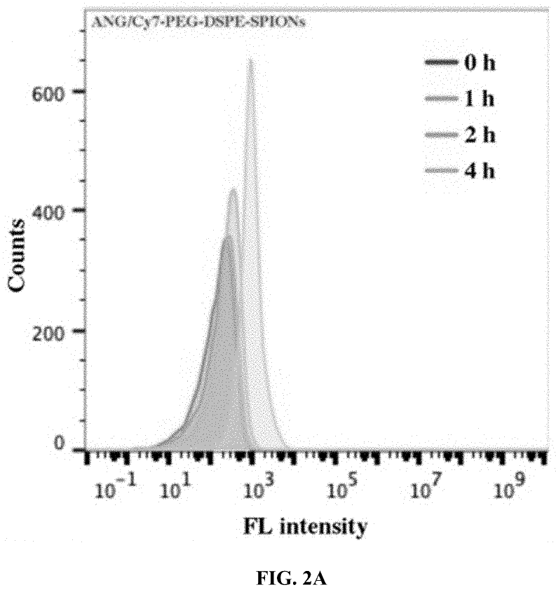

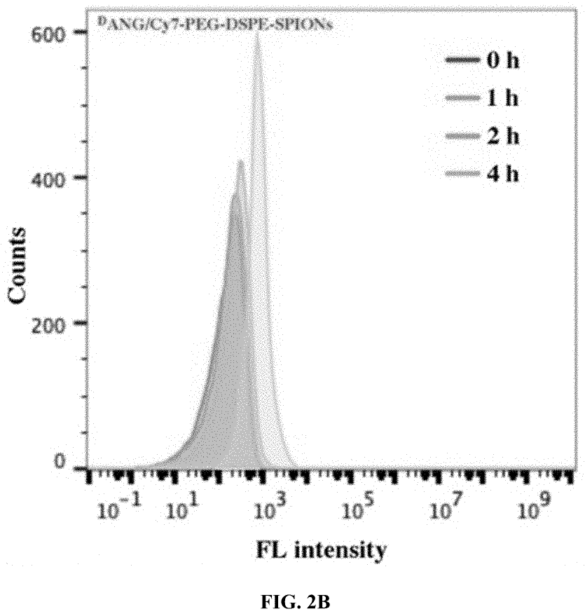

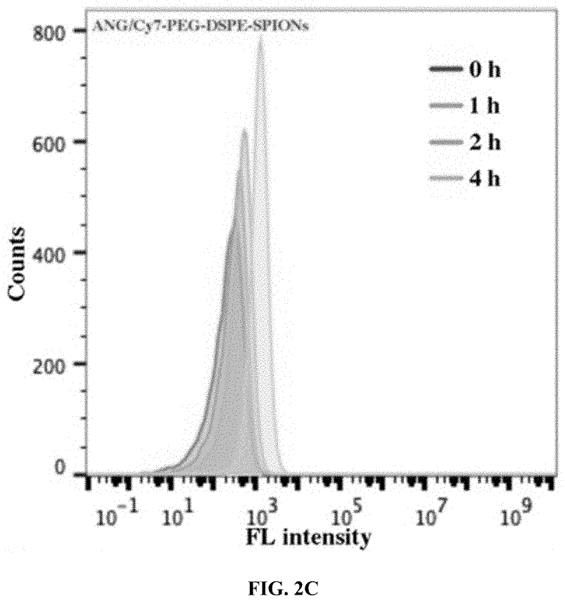

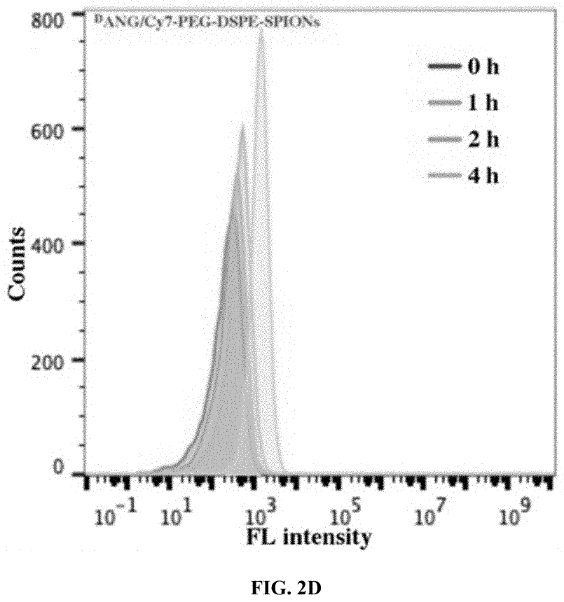

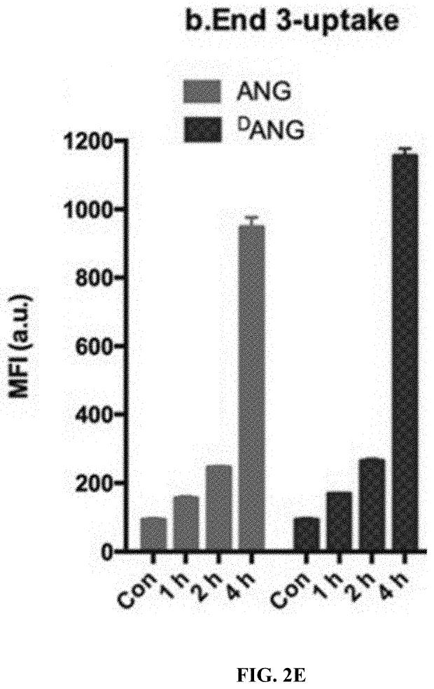

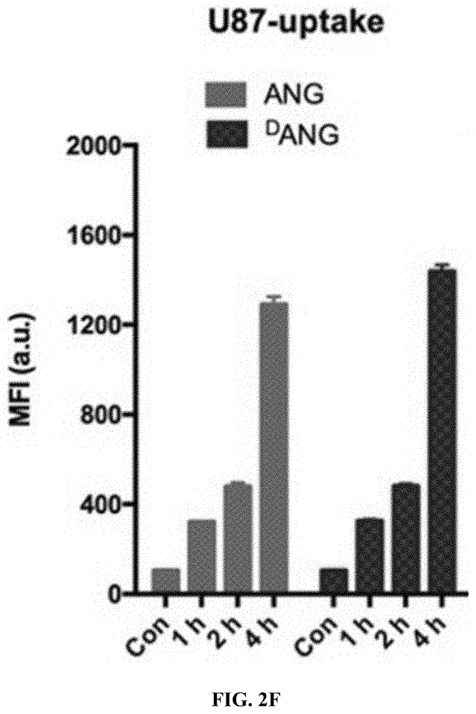

Experimental results: two kinds of targeting polypeptide modified probes (Peptides/Cy7-PEG-DSPE-SPIONs) are incubated with bEnd.3 and U87-MG cells for 1, 2 and 4 hours respectively, and then the cells are collected for flow cytometry, and the results are shown in FIG. 2. The results show that the uptake of probes by the two kinds of cells increased with the extension of co-culture time, which indicates that the uptake of probes by bEnd.3 and U87-MG shows a time-effect relationship.

### EXAMPLE 4

In this Example, the uptake of different probes by bEnd.3 cells and U87-MG cells is qualitatively observed.

Experimental method: cell plating: U87-MG cells and bEnd.3 cells were cultured with high glucose DMEM containing 10% fetal bovine serum and 1% penicillin/streptomycin at 37° C. and 5% CO₂. The round glass plate was sterilized and placed in a 24-well plate. When the cells grew well, the two kinds of cells were spread in the well plate, with a density of 2.5×10⁴ cells per well.

Probes pre-incubation: in order to explore the stability of functional polypeptides, probes were pre-incubated with human serum in a ratio of 1:1 for 3 h (80 rpm) at 37° C. before cell uptake.

Cell uptake: after the cells adhered to the wall, two probe solutions (Peptides/Cy7-PEG-DSPE-SPIONs) with a concentration of 50 μg/mL(Fe₃O₄) and two probe solutions pre-incubated with serum (pre-incubation Peptides/Cy7-PEG-DSPE-SPIONs) were added to continue culturing for 2 h and 4 h respectively, and the culture medium was removed at the set time and washed with PBS for 3 times, and then 4% paraformaldehyde was added to fix the cells for 30 min. After washing with PBS for 3 times, DAPI was added to stain the nucleus, and a sample preparation after washing was obtained for microscopy and pictures were taken with an optical microscope.

Experimental results: The uptake of probes modified by two polypeptides and probes preincubated by serum was observed by optical fluorescence microscope. The ANG/Cy7-PEG-DSPE-SPIONs and ^{D}ANG/Cy7-PEG-DSPE-SPIONs probes were incubated with human serum at a ratio of 1:1 for 3 h, and then co-cultured with cells for 2 h and 4 h. The experimental results show that both groups of polypeptide modified probes and their pretreatment groups could be effectively absorbed by cells and distributed in cytoplasm, and the red fluorescence signal of probes pretreated by serum under the same exposure conditions is significantly reduced compared with that of untreated groups. Compared with ^{D}ANG probe, the uptake of ANG polypeptide group probe is relatively small after serum incubation, which leads to a significant decrease in fluorescence, and this result is more obvious in U87-MG cell group. Therefore, in Example 5, the uptake of U87-MG cells to two groups of probes after serum co-incubation will be further quantitatively investigated by flow cytometry.

### EXAMPLE 5

In this Example, the uptake of two probes by U87-MG cells after pre-incubation with serum was quantitatively analyzed.

Experimental method: Cell plating: U87-MG cells were used to evaluate and analyze the uptake of two probes modified with targeting polypeptide and probes preincubated with serum. U87-MG cells were cultured in high glucose DMEM containing 10% fetal bovine serum and 1% penicillin/streptomycin at 37° C. and 5% CO₂. When the cells grew well, they were spread in a 12-well plate with a density of 1×10⁵ cells per well.

Flow cytometry: after the cells adhered to the wall, two probe solutions (Peptides/Cy7-PEG-DSPE-SPIONs) with a concentration of 50 μg/mL(Fe₃O₄) and two probe solutions (Pre-incubation Peptides/Cy7-PEG-DSPE-SPIONs) pre-incubated with serum were added respectively. The cells were cultured at 37° C. and 5% CO₂ for 4 h, and then collected for flow cytometry. The detection channel was set as APC-A750, and the data was processed and analyzed by flow cytometry software Flowjo.

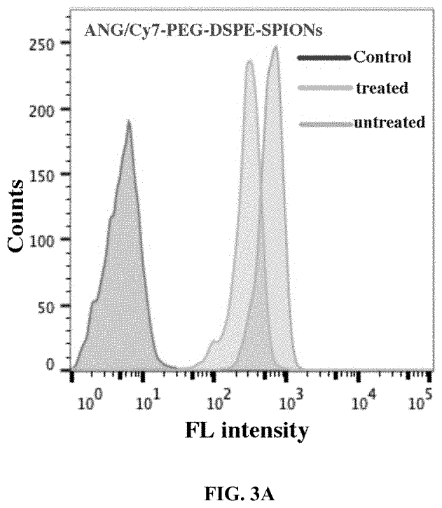

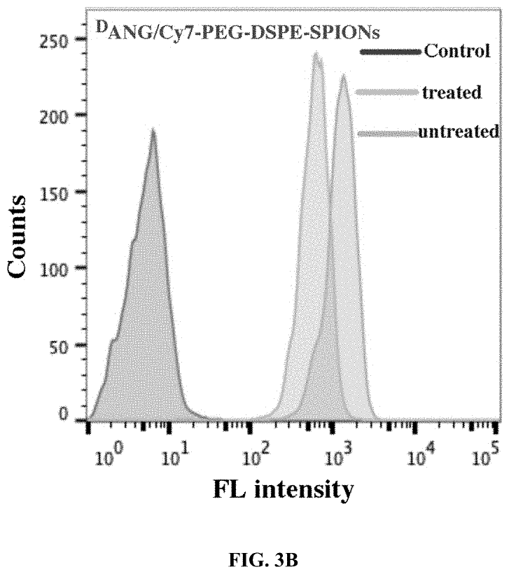

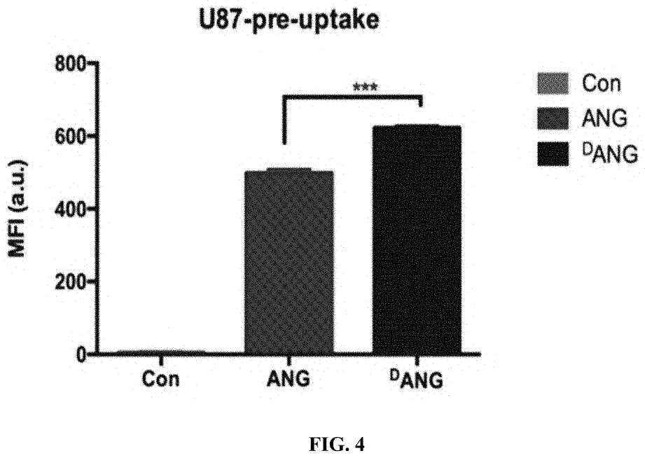

Experimental results: Because the modified polypeptide has targeting function, it can mediate the uptake of nanoprobes by cells. If the function is destroyed, the uptake of nanoprobes by cells will decrease significantly. By studying the uptake of probe by cells before and after co-incubation of human serum and peptide modified probe, we can evaluate the stability of peptide modified probe. U87-MG cells were co-cultured with probes of each group for 4 h, and the cells were collected for flow cytometry, as shown in FIG. 3. The quantitative results of uptake of ANG/Cy7-PEG-SPIONs and ^{D}ANG/Cy7-PEG-SPIONs by U87-MG cells after incubation with serum are shown in FIG. 4. The results show that the uptake of probes decreases after serum pretreatment, especially in ANG group. The stability of probes modified by ^{D}ANG group polypeptide is better than that of ANG group probes, which also confirms the above fluorescence observation results.

### EXAMPLE 6

This Example illustrates the construction and screening of Luc-U87-MG cells.

Experimental method: in order to determine whether the establishment of in situ glioblastoma model is successful or not and evaluate the fluorescence imaging effect, U87-MG cells transfected with Luciferase gene were used in the establishment of animal model in this example. The plasmid has resistance to puromycin. U87-MG cells were inoculated in a 6-well plate and cultured in high glucose DMEM containing 10% fetal bovine serum and 1% penicillin/streptomycin at 37° C. and 5% CO₂. When the cells grew to logarithmic phase, 10 μL virus concentrated solution (4.85×10⁸ cells/mL) and 2 μL infectious agent Polybrene (10 μg/mL) were added, and the cells were continued to culture for 48 hours. The Luc-U87-MG mixed clonal cells were screened by changing the screening medium with purine concentration of 1.5 μg/mL, and then Luc-U87-MG monoclonal cells were further selected by limited dilution method. After digestion, the cells were continuously diluted 10 times and inoculated into the 96-well plate, and finally was about 1-1.5 cells for each well. After the cell growth density reached about 60%, 100 μL of fluorescein potassium salt (1 mg/mL) was added and co-incubated for 2 min. The fluorescence signal was detected by the small animal living imager, which indicated that Luc-U87-MG single cell clones were obtained.

Experimental results: because the in situ glioblastoma is located in the brain, whether the tumor cells are successfully inoculated or not, the growth and size of the tumor cannot be observed intuitively. In order to better judge whether the model is successfully constructed for further imaging research, U87-MG cells expressing Luciferase were used as the inoculated cells. Firstly, luciferase gene was transferred into U87-MG cells by lentivirus packaging plasmid. Single cell clones with stable expression were screened by purine resistance gene on plasmid to obtain monoclonal Luc-U87-MG cell strain. The obtained cell strain reacts with fluorescein and is detected by small animal living imager.

### EXAMPLE 7

In this Example, that establishment of Luc-U87-MG in situ glioblastoma model in nude mice is described.

Experimental method: BAL B/c nude mice aged 5-6 weeks and weighing about 20 g were selected to establish the model. Firstly, Luc-U87-MG cells were cultured with high glucose DMEM containing 10% fetal bovine serum and 1% penicillin/streptomycin at 37° C. and 5% CO₂. When the cells were in good growth condition, the cells were digested by trypsin and collected for in situ inoculation of glioblastoma. The concentration of inoculated cells in each mouse was 5×10⁴ (5 μL single cell suspension). The anesthetic pentobarbital sodium (60 μg/kg) was injected according to the body weight of mice. After the mice were completely anesthetized, the mice were fixed on the stereotaxic instrument. After disinfection with iodine and alcohol, the skin of the head was cut longitudinally to expose the skull. The bregma as taken as the origin, a drilling hole was located 2 mm to the right and 1 mm to the front. Luc-U87-MG cell suspension was absorbed with a 5 μL microinjector, and the needle was 3mm deep the drill and retracted 1 mm, and the cells were injected at a rate of 0.5 μL/min. After finishing the injections, suspended the needle for 10 min and withdrew the needle. After the wound was disinfected, the skin was sutured with medical suture and disinfected, and the state of mice was continuously observed after operation. Three weeks after operation, the model was observed and confirmed to be successful with a small animal living imager.

Experimental results: Luc-U87-MG cells are inoculated in situ for 3 weeks, and the success of inoculation was detected by small animal living imager. The tumor cells of mice that are not successfully inoculated are not concentrated at the intracranial injection site and transferred below the head and neck (as shown in FIG. 5(A)), while nude mice that are successfully inoculated could observe that the tumor cells all gathered at the intracranial injection site and formed tumors (as shown in FIG. 5(B), FIG. 5(C)) and FIG. 5(D)).

### EXAMPLE 8

In this Example, T₂ weighted imaging and SWI(T₂*) imaging of magnetic resonance in vivo will be described.

Experimental method: nude mice bearing glioblastoma in situ were selected for MRI study. The magnetic resonance imaging was performed with Siemens Magnetom Trio 3.0 T magnetic resonance instrument and mouse coil. Imaging animals were divided into three groups, with three animals in each group:

Negative control group (group 1): non-targeted probe Cy7-PEG-DSPE-SPIONs was injected through tail vein at a dose of 10 mg Fe₃O_{4/}kg;

experimental imaging group (group 2 and group 3): targeting probes ANG/Cy7-PEG-DSPE-SPIONs and ^{D}ANG/Cy7-PEG-DSPE-SPIONs were injected through tail vein at the dose of 10 mg Fe₃O_{4/}kg respectively. After anesthetizing with isoflurane, the rats were scanned for the first time and the images were collected. And then, the probes were injected, and enhanced scanning was performed and images were collected. The time points were 0 h, 2 h, 4 h and 24 h. Scan parameters:

T₂ weighted imaging: TE=93 ms; TR=3000 ms; Thickness of layer (SL)=1 mm; Visual field (FoV)=66 mm×66 mm; Matrix size=256×256, NEX: 5;

SWI (T₂*) imaging: TE=20 ms; TR=32 ms; Thickness of layer (SL) =1mm; The Visual field (FoV)=50 mmx 50 mm; Matrix size=320×320, NEX: 2.

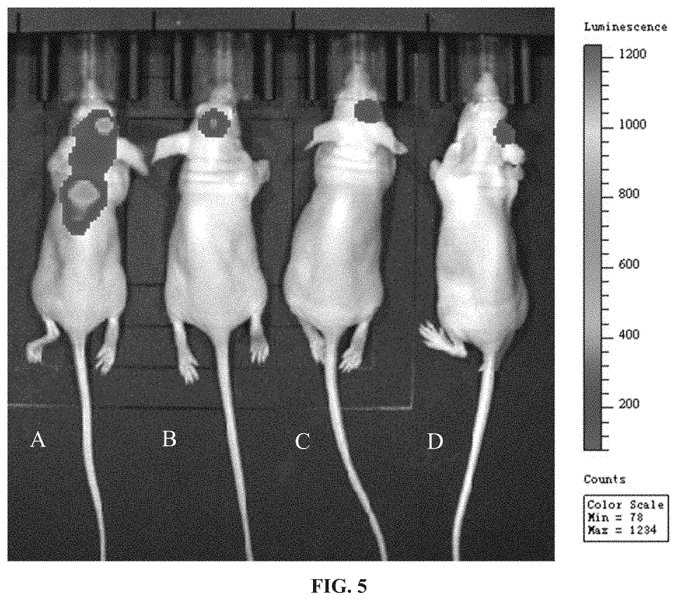

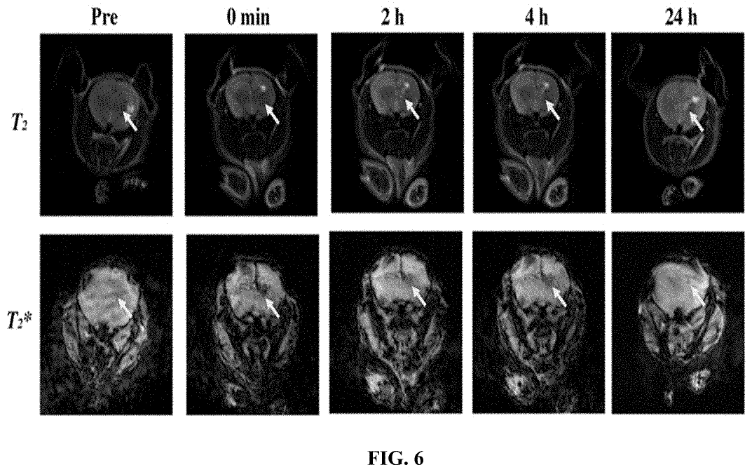

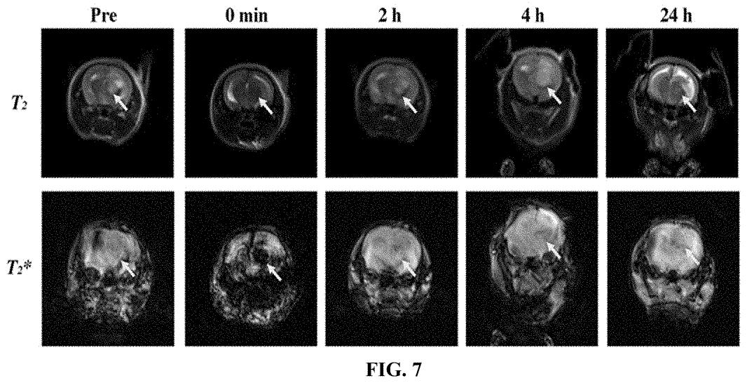

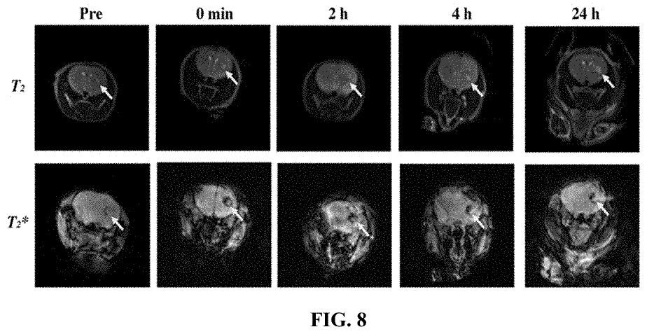

Experimental results: nude mice successfully established in situ glioblastoma were selected for in vivo imaging. Tumor-bearing nude mice were injected with three kinds of probes and carried out T₂ enhanced scanning, and the results show that Cy7-PEG-DSPE-SPIONs group cannot effectively enhance the image of tumor site on T₂ imaging (as shown in FIG. 6, T₂), after injection of the other two groups of probes (ANG/Cy7-PEG-DSPE-SPIONs, ^{D}ANG/Cy7-PEG-DSPE-SPIONs), the tumor site can be observed darker on T₂ imaging (FIG. 7, FIG. 8, T₂), and their enhancement effect decreases over time. However, due to the bright signal of tumor itself and the dark signal of normal brain tissue during T₂ plain scan, negative contrast agents have obvious disadvantages in enhancing T₂ of intracranial tumors, and enhanced scan weakens the contrast between normal tissues and tumor tissues.

Magnetic susceptibility weighted imaging (SWI, T₂*) is a magnetic resonance imaging technique that uses the different magnetic sensitivities of tissues to image. It is very sensitive to local magnetic field changes and shows low signal on images. Because iron deposition can cause the change of magnetic field, SWI technique is used to detect brain tumors using targeting peptide modified SPIONs probe, which is compared with T₂ weighted imaging. The results show that SWI can clearly outline the tumor and enhance the contrast between normal tissues and tumor tissues (FIGS. 6-8, T₂*).

0 min after SWI injection (in practice, scanning a T₂ sequence takes 4.5 min and scanning a SWI sequence takes 7.5 min), obvious signal changes in tumor part can be seen, and the dark signal will gradually decrease with time. After 24 h, the aggregation of SPIONs can still be detected by SWI sequence scanning, while T₂ weighted imaging can not be monitored for a long time. Compared with the probes without targeting modification, the probes modified by two polypeptides have stronger targeting imaging ability and aggregation ability. In addition, compared with the probe of ^{D}ANG polypeptide, the probe of ANG polypeptide group has weaker long-term imaging monitoring ability. As shown in FIG. 6, Cy7-PEG-DSPE-SPIONs group has low tumor aggregation degree in mouse brain and poor contrast imaging effect. In ANG/Cy7-PEG-DSPE-SPIONs group, the site of glioblastoma in mouse brain can darken rapidly in a short time, and the signal gradually disappears (FIG. 7) after a long time (for example, 24 h). On the contrary, the retention of probe can be detected in the site of glioblastoma in nude mice injected with ^{D}ANG polypeptide group for a long time (FIG. 8).

### EXAMPLE 9

This example illustrates fluorescence imaging of isolated organs.

Experimental method: In vivo imaging of small animals was used to conduct bioluminescence imaging and near-infrared fluorescence imaging of tumor-bearing nude mice successfully established in situ glioblastoma. Near infrared fluorescence imaging of intracranial tumors needed craniotomy, so that in vitro imaging was selected. Experimental grouping was the same as Example 8, which was divided into 3 groups. After the tail vein material was injected for 24 hours, a certain amount of fluorescein potassium salt solution (150 mg/kg) was injected intraperitoneally. After 15 minutes of reaction, the experimental animals were perfused with normal saline and 4% paraformaldehyde, and then their brain tissues and other main organs (heart, liver, spleen, lung and kidney) were taken. Subsequently, a small animal in vivo imaging system was used to observe the in situ visualization of glioblastoma and the distribution of materials in various organs (excitation wavelength was at 720 nm for near-infrared fluorescence imaging for detection wavelength at 820 nm).

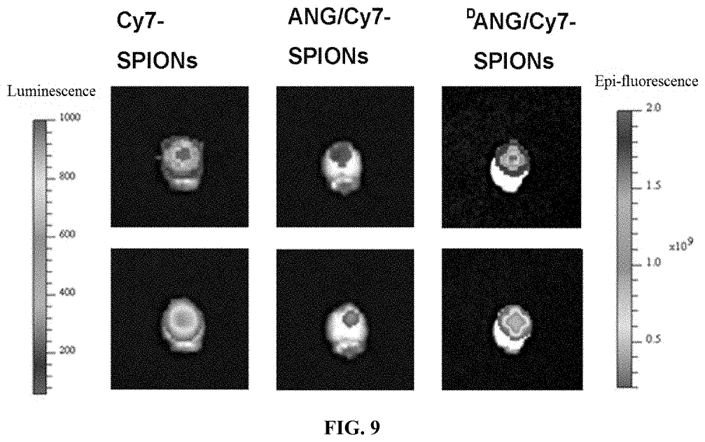

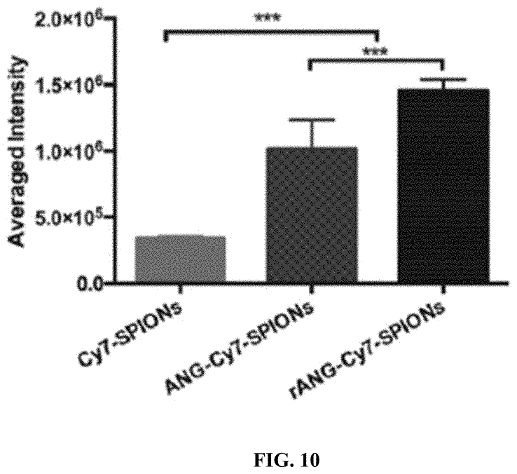

Experimental results: The small animal living imager was used for in vitro fluorescence imaging to evaluate the near infrared fluorescence imaging effect of the probe. Cy7-PEG-DSPE-SPIONs probe (control group) and two kinds of Peptides/Cy7-PEG-DSPE-SPIONs probes (experimental group) were injected into tumor-bearing nude mice for 24 h, then the size of glioblastoma in different groups was detected by bioluminescence imaging (BLI), and the aggregation amount of probes in different groups at tumor position was detected by near infrared fluorescence imaging, and the results are shown in FIG. 9. Finally, the average fluorescence intensity (total fluorescence intensity/bioluminescent photon number) was calculated to evaluate the average aggregation amount of the probe at the tumor site. The results show that the aggregation degree of Cy7-PEG-DSPE-SPIONs probe group is the weakest in tumor site, which is significantly different from that of polypeptide targeting group (p<0.05). In addition, the average fluorescence intensity of ANG/Cy7-PEG-DSPE-SPIONs probe group is significantly lower than that of ^{D}ANG/Cy7-PEG-DSPE-SPIONs probe group, and the difference is significant (p<0.05), as shown in FIG. 10.

### EXAMPLE 10

In this Example, the localization of the probe in the tumor-bearing brain will be explained.

Experimental method: after scanning, the experimental animals were perfused with physiological saline and 4% paraformaldehyde, and then their brain tissues were taken and fixed with stationary liquid for 2 days, then transferred to 30% sucrose solution for dehydration for 3 times. After complete dehydration, the brain tissues were embedded with OCT embedding agent and placed in a constant cold microtome for frozen section, while the sagittal position of the brain was kept parallel to the blade, with a thickness of 8 pm. The slices were placed on a cationic anti-dropping glass slide, the slices were dried and rinsed with clean water for 2 minutes, and then rinsed with ultrapure water for 2 minutes. The equal volume Prussian blue solution A (2% HCl solution) and solution B (2% potassium ferrocyanide solution) were mixed to prepare dye solution, and the tissue sections were stained and incubated for 30 min, then washed with ultrapure water twice for 3 min each time. The counterstain solution (2% nuclear fast red dye) was added to dye for 1 min. After dyeing, the tissue sections were washed with pure water, dried, sealed with neutral resin, and the specific distribution of probes were observed in the brain with optical microscope.

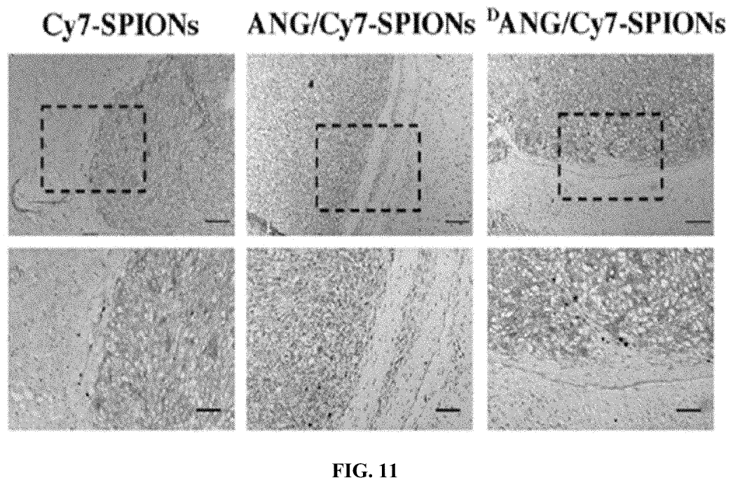

Experimental results: After the fixed brain tissue was further sectioned, Prussian blue staining was used to detect the distribution of probes in the brain and tumor location. According to staining analysis, Cy7-PEG-DSPE-SPIONs probe and two kinds of Peptides/Cy7-PEG-DSPE-SPIONs probes were injected into tumor-bearing nude mice for 24 h and then gathered at the edge of tumor, as shown in FIG. 11. It can also be observed from FIG. 11 that the aggregation degree of the two probes modified by polypeptide group is higher than that of the control group. The results are consistent with those of MR imaging, and the probe can effectively gather at the tumor boundary.

### EXAMPLE 11

This example illustrates the tissue distribution of probes in tumor-bearing mice.

Experimental methods: healthy BAL B/c mice were randomly divided into 4 groups, which were divided into normal saline control group and experimental group (Cy7-PEG-DSPE-SPIONs,ANG/Cy7-PEG-DSPE-SPIONs, ANG/Cy7-PEG-DSPE-SPIONs), with 3 mice in each group. 10 mg Fe₃O_{4/}kg probe solution or equal volume of normal saline was injected into the corresponding mice through tail vein, and then the mice were killed after 24 hours. The organs (brain, heart, liver, spleen, lung and kidney) of 2 mice in each group were randomly taken and fixed, and 0.2 g of each organ was cut into pieces, and then digested with 1 mL hydrogen peroxide and 2 mL nitric acid for two days, and then bathed in oil at 120° C. until no precipitate existed. After evaporating to dryness, 5 mL of 2% HNO₃ solution was added to a constant volume. After ultrasonic treatment for 20 minutes, 4 mL of the solution was taken into the EP tube and the Fe content in which was determined by ICP-MS.

Experimental results: The distribution and content of probes in main organs in vivo was determined by ICP-MS and fluorescence analysis. The main organs (heart, liver, spleen, lung, kidney, brain) of mice were perfused 24 h after injection of probe, and fluorescence imaging was performed in vitro with small animal living imager. The results of total fluorescence intensity analysis show that nanoprobes are mainly concentrated in liver and kidney. Through further quantitative analysis by ICP-MS, although the total amount of nanoprobes is obviously aggregated in liver and kidney, the Fe mass ratio of spleen is the highest, which may be related to its role as an iron metabolism organ.

### EXAMPLE 12

In this Example, the toxic and side effects of the dual-modality nanoprobe of the present disclosure on cells are explained.

Experimental method: U87-MG cells and HUVEC cells were cultured in high glucose DMEM containing 10% fetal bovine serum and 1% penicillin/streptomycin at 37° C. and 5% CO₂. In vitro cytotoxicity of Peptides/Cy7-PEG-DSPE-SPIONs was analyzed by CCK-8 kit. U87-MG cells and HUVEC cells were inoculated into 96-well plates according to 1×10⁴ cells per well. After culturing for 24 hours, probe medium containing Fe concentrations of 0.000, 3.125, 6.250, 12.500, 25.000 and 50.000 μg/mL were added to continue culturing for 24 hours. Then the plate was washed twice with PBS, and 100 μL CCK-8 reagent (stock solution: 10 μL, culture medium: 90 μL) was added to each well. After incubation for 1.5 h, the absorbance at 450 nm was detected by flow cytometry.

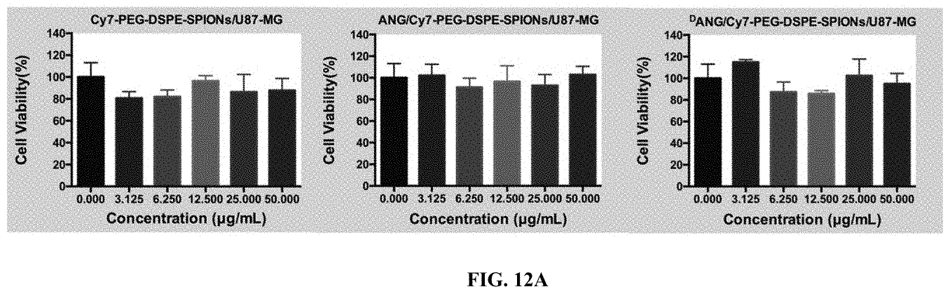

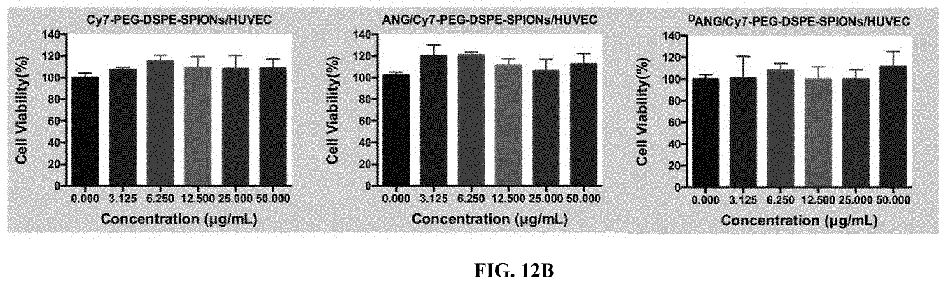

Experimental results: By studying the effect of Peptides/Cy7-PEG-DSPE-SPIONs on the proliferation of glioblastoma cells (U87-MG) and normal endothelial cells (HUVEC), the toxic and side effects on cells are evaluated. Two kinds of Peptides/Cy7-PEG-DSPE-SPIONs probes with different concentrations are added into cells, and after incubation for 24 h, their effects on cell proliferation are detected by CCK-8 kit. As shown in FIG. 12, in the range of 0-50 g Fe₃O_{4/}ml, the proliferation ability of U87-MG/HUVEC co-incubated with probes has no significant difference compared with the blank group (p>0.05), which indicates that the dual-modality nanoprobe of the present disclosure has no obvious toxic and side effects on cells.

### EXAMPLE 13

In this Example, the preliminary safety evaluation in vivo of the dual-modality nanoprobe of the present disclosure is explained.

Experimental method: healthy BAL B/c mice were randomly divided into 4 groups, which were divided into normal saline (control) group and experimental group (Cy7-PEG-DSPE-SPIONs, ANG/Cy7-PEG-DSPE-SPIONs, ^{D}ANG/Cy7-PEG-DSPE-SPIONs), with 9 mice in each group. According to the dosage of 10 mg Fe₃O₄/kg, the probe solution or equal volume of normal saline was injected into mice through tail vein. On the 1st, 3rd and 7th day after injection, 3 mice in each group were killed, and their organs (heart, liver, spleen, lung and kidney) were fixed. Then paraffin sections were made, hematoxylin-eosin (HE) staining was performed, and histopathological observation was carried out with optical microscope.

Experimental results: the experimental results are shown in FIG. 13-FIG. 15. Three kinds of probes (Cy7-PEG-DSPE-SPIONs, ANG/Cy7-PEG-DSPE-SPIONs, ^{D}ANG/Cy7-PEG-DSPE-SPIONs) are injected into tail vein of mice. After 1, 3, and 7 days, the results of HE staining sections of the main organs (heart, liver, spleen, lung, and kidney) of different groups can be analyzed. Compared with the control group, the experimental group injected with different probes has no obvious pathological features, which indicates that the various probes constructed do not have long-term systemic toxicity in vivo and have good safety in vivo.

The above are only specific embodiments of the present disclosure, but the scope of protection of the present disclosure is not limited thereto. Any person skilled in the art can easily think of changes or substitutions within the technical scope disclosed by the present disclosure. It should be covered within the protection scope of the present disclosure. Therefore, the protection scope of the present disclosure should be subject to the protection scope of the claims.

What is claimed is:

1. A dual-modality nanoprobe targeting glioblastoma, comprising: DSPE-PEG(2000)-Amine, superparamagnetic iron oxide nanoparticles (SPIONs), Cy7-NHS molecules, and a targeting polypeptide and/or enantiomer thereof.

2. The dual-modality nanoprobe targeting glioblastoma according to claim 1, wherein the targeting polypeptide is a targeting polypeptide ANG composed of L-type amino acids and the enantiomer of the targeting polypeptide is the enantiomer sequence ^{D}ANG of the targeting polypeptide composed of D-type amino acids.

3. The dual-modality nanoprobe targeting glioblastoma according to claim 2, wherein the targeting polypeptide ANG composed of L-type amino acids comprises the sequence of Ac-TFFYGGSRGKRNNFKTEEY-OH (SEQ ID NO: 1); the enantiomer sequence ^{D}ANG of the targeting polypeptide composed of D-type amino acids comprises the sequence of Ac-YEETKFNNRKGRSGGYFFT-OH (SEQ ID NO: 2).

4. The dual-modality nanoprobe targeting glioblastoma according to claim 1, wherein the dual-modality nanoprobe comprises Peptides/Cy7-PEG-DSPE-SPIONs, and the Peptides/Cy7-PEG-DSPE-SPIONs comprise ANG/Cy7-PEG-DSPE-SPIONs and/or ^{D}ANG/C_{y}7-PEG-DSPE-SPIONs.

5. The dual-modality nanoprobe targeting glioblastoma according to claim 4, wherein the dual-modality nanoprobe comprises ^{D}ANG/Cy7-PEG-DSPE-SPIONs.

6. A method of preparing a dual-modality nanoprobe targeting glioblastom, method comprising: using superparamagnetic iron oxide nanoparticles (SPIONs) as a core, modifying a surface of SPIONs with near infrared fluorescent molecule Cy7, and taking targeting polypeptide and/or enantiomer thereof as ligands to construct magnetic resonance/fluorescence dual-modality nanoprobe targeting glioblastoma.

7. The preparation method of the dual-modality nanoprobe targeting glioblastoma according to claim 6, comprising:
preparing SPIONs, coating DSPE-PEG(2000)-Amine molecules on the surface of the SPIONs through hydrophilic-hydrophobic interaction, and carrying out ultrasonic dispersion pretreatment on pegylated magnetic nanoparticles having amino groups to uniformly disperse the nanoparticles in an aqueous solution, replacing the aqueous solution of magnetic nanoparticles by 0.02 M, pH8 borate buffer solution with a 10 KDa ultrafiltration tube;
activating carboxyl groups of the targeting polypeptide, wherein the targeting polypeptide comprises a targeting polypeptide ANG composed of L-type amino acids and/or the enantiomer sequence ^{D}ANG of the targeting polypeptide composed of D-type amino acid;
placing the activated targeting polypeptide ANG and/or the enantiomer sequence ^{D}ANG of the targeting polypeptide composed of D-type amino acid and NH2-PEG-DSPE-SPIONs on a shaking table to react for 2 hours, and separating the unreacted targeting polypeptide by a 30 KDa ultrafiltration tube to obtain a pegylated magnetic nanoparticle Peptides/PEG-DSPE-SPIONs solution;
adding Cy7-NHS molecules into the pegylated magnetic nanoparticle solution, and shaking overnight at 4° C. in the dark for incubation; after the incubation, removing the unreacted Cy7-NHS dye molecule by ultrafiltration tube to obtain a Peptides/Cy7-PEG-DSPE-SPIONs probe.

8. The preparation method of the dual-modality nanoprobe targeting glioblastoma according to claim 7, wherein SPIONs are synthesized by a hydrothermal method.

9. The preparation method of the dual-modality nanoprobe targeting glioblastoma according to claim 8, wherein the hydrothermal synthesis of SPIONs comprises:
mixing iron acetylacetonate, 1,2-hexanediol, oleic acid, oleamide and benzyl ether, passing argon to remove air and stirring to obtain a mixture;
heating the mixture to 200° C., keeping constant temperature for 2 h, then heating to 300° C., refluxing for 1 h to obtain a black mixture;
cooling the black mixture to room temperature, precipitating a product with ethanol, centrifuging to remove solvent, and dispersing the product to hexane to obtain a SPIONs solution; and
centrifuging the SPIONs solution to remove aggregates.

10. The preparation method of the dual-modality nanoprobe targeting glioblastoma according to claim 7, wherein activating the carboxyl groups of the targeting polypeptide ANG composed of L-type amino acids and/or the enantiomer sequence ^{D}ANG of the targeting polypeptide composed of D-type amino acid comprises:
dissolving the targeting polypeptide ANG composed of L-type amino acids and/or the enantiomer sequence ^{D}ANG of the targeting polypeptide composed of D-type amino acid with 0.02 M MES buffer solution with a pH of 5.5;
adding EDC molecules and NHS molecules, reacting for 25 min at 25° C. and 180 rpm in a constant temperature shaker to activate carboxyl groups of the targeting polypeptide sequence;
removing unreacted EDC and NHS with 2 KDa ultrafiltration tube.

11. The dual-modality nanoprobe targeting glioblastoma according to claim 4, wherein the targeting polypeptide is a targeting polypeptide ANG composed of L-type amino acids; the enantiomer of the targeting polypeptide is the enantiomer sequence ^{D}ANG of the targeting polypeptide composed of D-type amino acids.

12. The dual-modality nanoprobe targeting glioblastoma according to claim 4, wherein the targeting polypeptide comprises a targeting polypeptide ANG composed of L-type amino acids comprising a sequence of Ac-TFFYGGSRGKRNNFKTEEY-OH (SEQ ID NO: 1);
and/or an enantiomer sequence ^{D}ANG of the targeting polypeptide composed of D-type amino acids comprising a sequence of Ac-YEETKFNNRKGRSGGYFFT-OH (SEQ ID NO: 2).
